(19) United States
(12) Patent Application Publication
(10) Pub. No.: US 2013/0345653 A1
(43) Pub. Date: Dec. 26, 2013

(54) CATHETERS

(71) Applicant: Roger Charles Leslie FENELEY, Bristol (GB)

(72) Inventor: Roger Charles Leslie FENELEY, Bristol (GB)

(73) Assignee: ALTERNATIVE UROLOGICAL CATHETERS LIMITED, Bristol (GB)

(21) Appl. No.: 13/985,771

(22) PCT Filed: Feb. 8, 2012
(86) PCT No.: PCT/GB2012/000132

§ 371 Date: Sep. 4, 2013

(30) Foreign Application Priority Data
Feb. 16, 2011 (GB) 1102731.5

(51) Int. Cl. A61F 5/44

(52) U.S. Cl. CPC A61F 5/4405; A61F 5/4408
USPC 604/328

(57) ABSTRACT

A catheter for use in a urine collection system has a hollow tube forming a suprapubic section (1), a bladder section (2) and a urethral section (3). There are drainage holes in the bladder section (2) and optionally either or both of an abdominal stop (5) and a non-return cuff (6) on the bladder section (2) which holds the catheter in place. There may also be a valved drainage section (31) extruding from the suprapubic section (1) to a drainage outlet (32), or a urine collection bag (35). The drainage section (31) and/or the suprapubic section (1) may have a pH sensor therein. There may also be a urine collection container (12) connected to the suprapubic section (1) the connection forming a closed valve-free fluid flowpath from the bladder section to the urine collection container and from the urine collection container to the bladder section, whereby the urine collection container provides a low pressure reservoir.

### BACKGROUND OF THE INVENTION

1. Field of Invention

The present invention relates to catheters for drainage of the urinary bladder. In particular, the invention relates to suprapubic urethral catheters and catheter systems.

2. Summary of the Prior Art

Urinary catheterisation introduces major medical, social and economic issues. The catheter is a medical device used to drain urine from the bladder but it also provides a conduit for bacteria to enter the body. One out of 4 hospitalized patients receives an indwelling catheter [1]. Catheter-associated urinary tract infections present the most common nosocomial infections, developing at an estimated rate of 5% per day and increasing the risk of bacteraemia and mortality [2]. Financial estimates in 2000 suggested that a symptomatic urinary tract infection raises the cost of care by around £420 and bacteraemia to at least £1750 by lengthening the stay in hospital and antibiotic therapy [3]. In developing countries the rates of healthcare-associated infections are 3 to 5 times higher than international standards [4] The self-retaining urinary catheter introduced by Dr Foley in 1937 has been the standard product in routine use for over 70 years [5]. The catheter is retained in the bladder by means of an inflatable balloon but this prevents the bladder from emptying completely, leaving residual infected urine. Long-term urinary catheterisation (LTC) provides a well-established method of management, draining urine from the bladder into a urine collection bag. LTC carries a high risk of complications; catheter-associated complications occur in over 70% of patients, the majority resulting from the rapid bacterial invasion of the catheter [6]. Urinary tract infection can lead to life threatening septicaemia but the common problems of encrustation and blockage of the catheter, urinary leakages and discomfort from the catheter have an immense impact not only on an individual's quality of life but on the costs to healthcare services [7]. The prevalence of LTC increases with age and although considered a last resort, many thousands of people rely on this method of urinary collection because no acceptable alternative is available [8].

A Foley-type catheter is usually positioned in a patient by passing it up through the urethra. Such a catheter is known as a urethral catheter. However, Foley-type catheters may alternatively be inserted into the bladder through the abdominal wall just above the pubic bone i.e. via a surgically produced suprapubic track. The latter is known as a suprapubic catheter.

Suprapubic urethral catheters are also known in the art. For example, see GB2343847A (also by the present inventor). A suprapubic urethral catheter is positioned in a patient via a suprapubic track and enables either suprapubic or urethral drainage of a urinary bladder. A suprapubic urethral catheter generally consists of a tube having, sequentially, a suprapubic section that will be located suprapubically in a patient, a bladder section that will be located within the bladder of the patient, and a urethral section that will be located within the urethra of the patient.

### SUMMARY OF THE INVENTION

The present invention aims to overcome at least some of the aforementioned deficiencies in conventional self-retaining Foley-type catheters.

The present invention has been developed particularly for those patients who are experiencing complications with the conventional self-retaining Foley-type catheter and urine collection bag. Recurrent blocking of the catheter and bypassing of urine around the catheter are common problems associated with long-term urinary catheterisation. In addition the present invention provides a versatile method of urinary drainage and collection and may be indicated in patients presenting specific needs for urine collection. A system that mimics the cyclical filling and emptying of the bladder has been advocated to reduce the risk of urinary infections and the incidence of catheter blockages [9]; the present invention has been designed to provide this.

The present invention is concerned with several developments of a catheter for use in a urine collection system, which developments may be used alone, or in combination. However, before describing those developments, it is necessary to describe the structures to which those developments will be applied. The structures to which the developments of the present invention will be applied are each referred to as a “starting point” for the present invention. In particular, two starting points will be described, and the developments of the present invention may be used with either or both starting point.

A first starting point for the present invention involves a self-retaining mechanism for a suprapubic urethral catheter having an arrangement for retaining the bladder section of the suprapubic urethral catheter in the bladder.

At its most general, this first starting point proposes that a non-return cuff is mounted on a section of a catheter which is to be inserted into a bladder. The non-return cuff is deformable towards the catheter so that it may be inserted into the bladder without significant resistance through the suprapubic tract in the abdominal and bladder walls (e.g. when the catheter is inserted from a suprapubic direction). The projecting cuff then may prevent or resist the catheter being withdrawn from the bladder in a suprapubic direction. The bladder section of the catheter will then normally have holes (e.g. drainage eyes) for ingress of urine to the catheter duct (which duct may be referred to as a drainage lumen).

This first starting point may then have a suprapubic urethral catheter comprising a hollow tube having sequentially a suprapubic section, a bladder section and a urethral section; wherein, there is an abdominal stop attached to the suprapubic section; and wherein, the bladder section comprises one or more drainage holes in the wall of the tube and there is a non-return cuff attached to the bladder section between the one or more drainage holes and the abdominal stop; and wherein, the non-return cuff has a projecting portion projecting outwardly from the tube which is deformable towards the tube and which resists deformation away from the tube.

The projecting portion projecting outwardly from the tube of the non-return cuff is deformable towards the tube, e.g. on insertion of the cuff into a suprapubic track in the abdominal wall of a user and into the bladder. However, once the cuff has entered the bladder, the projection portion springs outwardly once again and resists deformation away from the tube. Therefore if the catheter is urged in the retrograde direction, i.e. back out of the suprapubic track along which it was inserted, the cuff will not pass back through the entry aperture to the bladder and the cuff does not move relative to the bladder section, therefore the cuff prevents the bladder section from extruding retrogradely out of the bladder.

In other words, the non-return cuff is arranged on the bladder section of the catheter so that it is flexible enough to be inserted into the bladder via a suprapubic track in the abdominal wall of a user but the non-return cuff is stiff enough to prevent the bladder section from extruding retrogradely, i.e. backwards, out of the bladder along the suprapubic track.

Such a structure enables a catheter to be retained in the bladder without a balloon, enabling the bladder to fill and empty at low pressure and thus matching as closely as possible the normal physiological and mechanical characteristics of the normal bladder. The regular cycle of filling and emptying, together with the integrity of the urethral lining of the bladder, provides the chief defence mechanisms against the onset of urinary tract infection.

The non-return cuff may be made from any material having suitable properties (e.g. medical grade silicone), and arranged in any appropriate structural configuration to provide the necessary flexibility and stiffness. The non-return cuff may be fixed to the bladder section of a catheter using a suitable adhesive (e.g. a medical grade silicone glue), or may be integral with the bladder section.

Various shapes and structures for the non-return cuff are envisaged. For example, the projecting portion of the non-return cuff may comprise a wing, leg or arm, inclined away from the tube, wherein the end of the wing, leg or arm, furthest from the tube is nearest the abdominal stop. Alternatively, the projection portion may be of a frusto-conical shape.

Preferably, the projecting portion of the cuff comprises two inclined wings. More preferably the non-return cuff comprises a channel for receiving the tube of the catheter, and the two inclined wings are positioned on opposing sides of the channel. The two inclined wings may be joined by a resiliently deformable element and if so, the wings are biased apart from each other by the deformable element.

At the end of the wings furthest from the abdominal stop, the non-return cuff may have a sleeve formed around the channel and attached to the tube. In this embodiment, at the end of the sleeve furthest from the abdominal stop, the walls of the sleeve may taper towards the tube. This tapering is for easing insertion of the cuff into the suprapubic track and into the bladder.

Each wing may be shaped as a distorted elongated polygon, (preferably a hexagon), wherein the polygon is elongated along the length of the wing, and the widest part of the polygon is found towards the end of the wing being furthest from the abdominal stop. This shape balances the need for strength to resist deformation away from the tube with the need for ease of insertion.

Preferably the catheter comprises an irrigation hole in the wall of the tube positioned in the bladder section within the projecting portion of the non-return cuff. This enables any accumulated debris to be washed out.

Usually the catheter comprises a plurality of drainage holes or eyeholes located along the bladder section. Preferably at least one drainage hole, most preferably the drainage hole nearest the urethral section of the catheter (that is, furthest downstream) is retainable in a position at the lowest point in the bladder. This enables the bladder to be drained completely of urine, or allows only a minimal volume of residual urine to remain in the bladder on draining, and therefore reduces the risk of infection. One or more drainage holes may be positioned in the bladder section within the region of the non-return cuff to facilitate irrigation and to flush out any debris accumulated within it.

The abdominal stop, attached to the suprapubic section of the catheter, may be in the form of a flange. It may have a channel for receiving and gripping an external part of the suprapubic section of the catheter. Preferably the position of the abdominal stop along the catheter is adjustable, more preferably it is slidably adjustable. The channel through the flange preferably has an oval hole which can be released by simultaneous pressure on both lateral ends of the flange to allow adjustment of its position on the catheter.

Once the catheter is in situ in a patient, the abdominal stop remains external to the body. The position of the abdominal stop can then be adjusted and held against the abdominal wall to prevent the catheter from moving forwards out of the body via the urethra.

Use of the abdominal stop together with the non-return cuff means that in situ, both forward and retrograde movement of the catheter is prevented. Therefore the catheter is securely held in position. For example, this ensures that the drainage holes in the bladder section remain in an optimum position for maximum drainage of the bladder, and overall comfort for the patient may be improved.

There may be a marker at the junction between the bladder section and the urethral section of the catheter. Such a marker is identifiable when in situ in a patient by using e.g. radiological or ultrasound means. This helps to ensure the bladder section is implanted in the correct position in the patient or user.

In preferred arrangements, the catheter consists of a flexible silicone tube, 65-67 cm long and 12 or 14 FG/Ch size. The suprapubic section is preferably 20 cm in length. The upper end of the suprapubic section can be cut to a suitable length for the user.

The bladder section is provided in various lengths to fit different sizes of bladder. The preferred lengths for the bladder section are 5 cm and 7 cm.

The urethral section is preferably 40 cm in length but can be cut to a suitable length to fit into a drainage funnel or connector which can be fitted e.g. with a standard catheter valve. The wall of the tubing of this section of the catheter may be thinner than the other sections of the catheter. Thus the catheter can be used for draining urine from the bladder assisted by gravity by opening a manually operable catheter valve fitted to the drainage funnel or connector at the urethral end of the catheter.

A second starting point for the present invention involves a urine collection system having a low pressure reservoir which enables the functional capacity of the bladder to be increased. In other words an upstream overflow tank is provided to relieve pressure in the system.

In this second starting point, the overflow tank may then be in the form of a reservoir connected to a suprapubic section of a catheter which is to extend into the bladder of a patient. Thus, the second aspect of the invention may provide a urine collection system comprising:

a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, the connection forming a flowpath from the bladder section to the urine collection container and thus providing a low pressure reservoir.

If pressure in the bladder rises above a threshold pressure, urine flows upstream and enters the urine collection container and then returns to the bladder when the pressure in the bladder falls below the threshold. For example, if pressure in the bladder rises as a result of a contraction of the bladder (detrusor) muscle, urine enters the urine collection container and then returns to the bladder as the bladder muscle relaxes.

Many patients requiring long-term catheterization suffer from a neurological disease or injury which accounts for their loss of bladder control. Under these conditions, the bladder can become overactive with inappropriate uncontrolled contractions both of the bladder and the sphincter closing the urethral passage during filling. This is termed detrusor sphincter dysynergia. These contractions cause the pressure to rise in the bladder which in turn creates back-pressure on the kidney(s) with the risk of serious damage to their function. The objective of the upstream urine collection container is to obviate the pressure rise by providing a low pressure urinary reservoir.

Preferably, the urine collection container is attachable to the abdomen of a patient, more preferably via a waistband. Preferably the urine collection container is a pouch, most preferably it is an abdominal urine collection pouch.

The urine collection container will preferably hold a volume of e.g. up to 500 ml of urine. The urine collection container may also act as a portal for drug administration.

A Luer fitting may be included at the top of the urine collection container so that the container and the catheter can be irrigated if necessary to clear any accumulated debris. The system optionally includes provision for irrigating the system. For example, normal saline can be flushed through the system.

Having described the starting points of the present invention, the developments corresponding to the present invention will now be discussed. As mentioned previously, each of these developments may be applied to either starting point.

In a first development of the invention, the catheter has a drainage section which extends from the suprapubic section to a drainage outlet, and has at least one valve therein.

It has previously been mentioned that the urethral section may fit to a drainage funnel or connector which can be fitted with a standard catheter valve, but in this first development drainage is via the suprapubic section, and is valve-controlled. A drainage from the suprapubic section is preferred because of ease of use by the patient or carer.
Thus, in this first development there may be provided a suprapubic urethral catheter comprising:
a hollow tube having sequentially a suprapubic section,
a bladder section and a urethral section;
wherein, there is an abdominal stop attached to the suprapubic section; and
wherein, the bladder section comprises one or more drainage holes in the wall of the tube and there is a non-return cuff attached to the bladder section between the one or more drainage holes and the abdominal stop; and wherein the catheter further comprises a drainage section extending from the suprapubic section to a drainage outlet, the drainage section having at least one valve therein.
As in the first starting point, the non-return cuff may have a projecting portion projection outwardly from the tube which is deformable towards the tube and which resists deformation away from the tube.
Alternatively, the non-return cuff may be a Foley balloon.
There may also be provided a urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, providing a flowpath from the bladder section to the container, and thus providing a low pressure reservoir and wherein the catheter further comprises a drainage section extending from the suprapubic section to a drainage outlet, the drainage section having at least one valve therein.

The or each valve is preferably a pinch valve, as that reduces any possibility of contamination reaching the urine flow path of the catheter via the valve. The valve may have a movable actuator which, when the valve is a pinch valve, will compress the drainage section. To actuate that actuator, there may be a controller which moves the actuator at e.g. regular intervals to permit time duration. Such an arrangement is preferable when the user has a limited manual dexterity. Alternatively, the actuator may be controlled by a user-activatable control. Such a control may need to take into account the manual dexterity of the user. It may then be preferable to permit the user to control the or each valve remotely by a suitable remote signalling system, either magnetic or electromagnetic.

In a second development of the present invention, the drainage section has a urine collection bag. Such an arrangement will normally be used together with one or more valves, and it is preferable that there is then at least one valve between the suprapubic section and the urine collection valve, at least one further valve between the urine collection bag and the outlet of the drainage section. With such an arrangement, the bag may be allowed to fill by suitable control of the valve or valves between the suprapubic section and the collection bag, and the bag may then subsequently be drained via the further valve or valves. This may permit the system to mimic normal bladder action, by providing bladder drainage at regular intervals.

Thus, according to the second development there may be provided a suprapubic urethral catheter comprising:

a hollow tube having sequentially a suprapubic section, a bladder section and a urethral section;
wherein, there is an abdominal stop attached to the suprapubic section; and
wherein, the bladder section comprises one or more drainage holes in the wall of the tube and there is a non-return cuff attached to the bladder section between the one or more drainage holes and the abdominal stop; and wherein the catheter comprises a drainage section extending from the suprapubic section to a drainage outlet, the drainage section including a urine collection bag.

Again, as in the first starting point, the non-return cuff may have a projecting portion projecting outwardly from the tube which is deformable towards to tube and which resists deformation away from the tube.

Alternatively, the non-return cuff may be a Foley balloon.

There may also be provided a urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
   a urine collection container connected to the suprapubic section, providing a flowpath from the bladder section to the container, and thus providing a low pressure reservoir and wherein the catheter comprises a drainage section extending from the suprapubic section to a drainage outlet, the drainage section including a urine collection bag.

In the second development of the present invention, the urine collection bag may contain a pH sensor. Such a pH sensor may be e.g. as described in WO 2006/000764.

Alternatively the pH sensor may be in the drainage and/or suprapubic section of the catheter, or in the urine collection container connected to the suprapubic section. This represents a third aspect of the invention.

Thus, there may be provided a suprapubic urethral catheter comprising:
a hollow tube having sequentially a suprapubic section, a bladder section and a urethral section; wherein, there is an abdominal stop attached to the suprapubic section; and
wherein, the bladder section comprises one or more drainage holes in the wall of the tube and there is a non-return cuff attached to the bladder section between the one or more drainage holes and the abdominal stop; and wherein the catheter comprises a drainage section extending from the suprapubic section to a drainage outlet, and said drainage section and/or said suprapubic section has a pH sensor therein.

There may also be provided a urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
   a urine collection container connected to the suprapubic section, providing a flowpath from the bladder section to the container, and thus providing a low pressure reservoir;
wherein the catheter comprises a drainage section extending from the suprapubic section to a drainage outlet, and said drainage section and/or the suprapubic section has a pH sensor therein.
In a further development, being a fourth aspect of the invention, the urine collection pouch is protected by a protective cover.
Thus, there may be provided a urine collection system comprising:
   a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, providing a flowpath from the bladder section to the container, and thus providing a low pressure reservoir;
   wherein there in a protective cover over the urine collection container.

As mentioned above, the developments of the present invention may be used singularly, or in any combination.

There may also be a kit of parts for assembling a catheter or urine collection system as previously defined.

The invention includes any combination of the aspects and preferred features described herein except where such a combination is clearly impermissible or expressly avoided.

### BRIEF DESCRIPTION THE DRAWINGS

Embodiments of the present invention will now be described, by way of example, with reference to the accompanying diagrammatic drawings, in which:

FIG. 1 shows a part cut away view of a catheter used in the present invention.

FIG. 2 shows a part cut away view of the catheter of the FIG. 1.

FIG. 3 shows an abdominal stop that is used with the catheter of FIGS. 1 and 2.

FIGS. 4a to 4e show a non-return cuff for use with the catheter of FIGS. 1 to 3. FIG. 4a is a 3-D view; FIG. 4b is a longitudinal cross-sectional view; FIG. 4c is an enlarged view of the aspect “A” circled in FIG. 4b; FIG. 4d is a side view; and FIG. 4e is an end view.

FIGS. 5a and 5b show a catheter with a non-return cuff and the catheter is attached to an abdominal collection pouch. FIG. 5a shows a sagittal view of this embodiment in-situ in a male patient. FIG. 5b shows a frontal view of this embodiment in-situ in a male patient.

FIG. 6 shows schematically an embodiment of the present invention, in which the arrangements of FIGS. 1 to 5 further include a drainage section of the catheter.

FIGS. 7a and 7b show schematically a protective cover for a urine collection pouch which may be used in the present invention.

FIG. 8 shows an arrangement similar to FIG. 5b, but which uses a different catheter.

FIG. 9 shows a view similar to FIG. 8 but with a modified catheter.

### DETAILED DESCRIPTION

The following detailed description of some embodiments of the invention should be read with reference to the drawings, wherein like reference numerals indicate like elements throughout the several views.

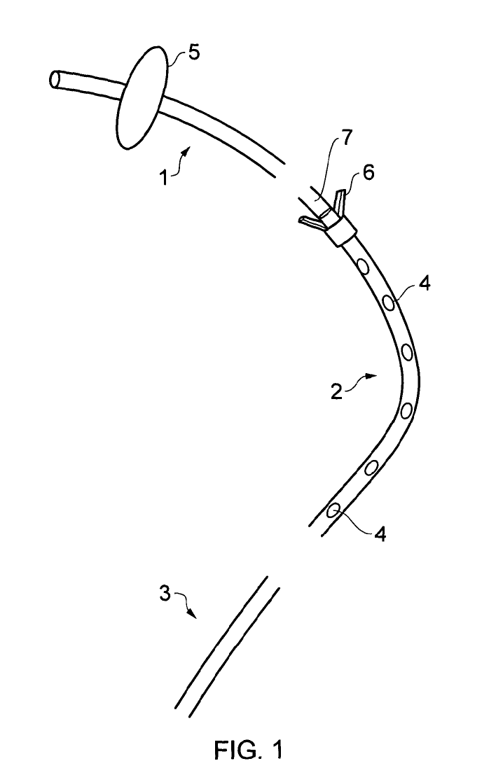

Referring to FIG. 1, there is illustrated a part cut away view of a suprapubic urethral catheter used in the present invention.

The catheter includes a flexible and hollow tube which may be described in three sections, namely a suprapubic section 1, a bladder section 2 and a urethral section 3. The tubing may be continuous, or there may be two or three sections of tubing joined together.

The suprapubic section 1 passes through an abdominal stop 5 (also referred to as an abdominal flange) which can be adjusted by sliding it along the catheter before the catheter enters the abdominal wall. The upper end of this section can be cut to a suitable length for the patient and can be fitted to an abdominal urine collection pouch or a drainage funnel or connector (not shown).

The bladder section 2 comes in various lengths depending on the size of the bladder. At its suprapubic or upper end it is fitted with a non-return cuff 6 that prevents the catheter from extruding retrogradely from the bladder and at its lower end, a radio-opaque, ultrasound or other marker (not shown in FIG. 1) denotes its junction with the urethral section. A plurality of drainage eyeholes 4 are placed along the whole length of this section which enables the bladder to be drained completely of urine towards the urethral or lower end assisted by gravity or if this is closed through the upper end into a urine collection pouch (not shown in FIG. 1).

The urethral section 3 is a tube which can be cut to length appropriate to the individual patient and fitted into a drainage funnel or connector (not shown in FIG. 1). A standard urethral valve (not shown in FIG. 1) which can be opened or closed can be inserted into the drainage funnel to store or empty urine from the bladder. A urine collection bag can be fitted to the catheter valve at the urethral end if necessary.

The urethral section may have an internal diameter of 3.7 mm and an external diameter of 4.7 mm, and the bladder and suprapubic sections may have an internal diameter of 3.1 mm and an external diameter of 4.7 mm. Thus the thicknesses of the bladder and suprapubic sections is then 0.8 mm and the wall thickness of the urethral section is 0.5 mm, so it is thinner than the wall thickness of the bladder and suprapubic sections.

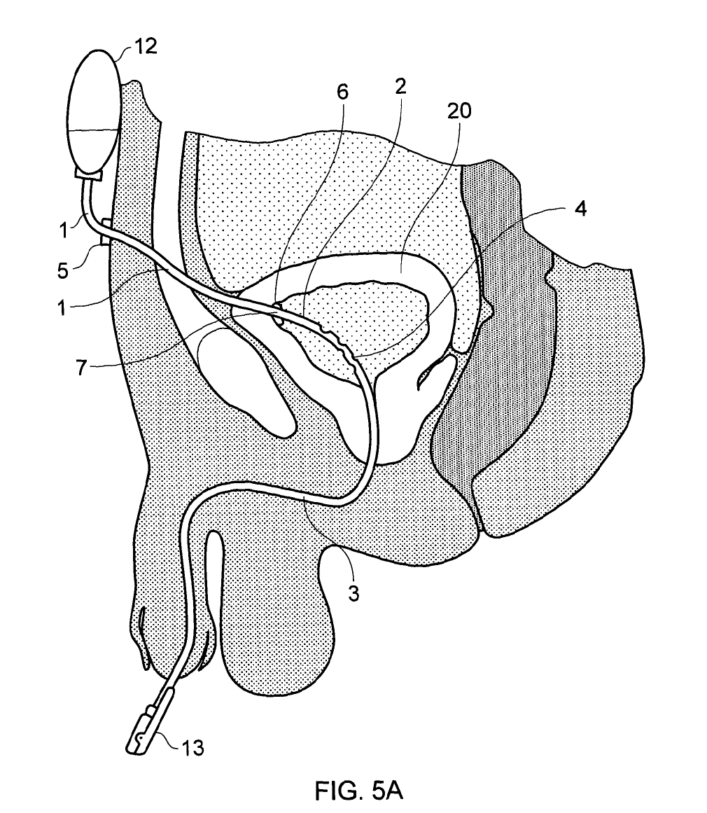

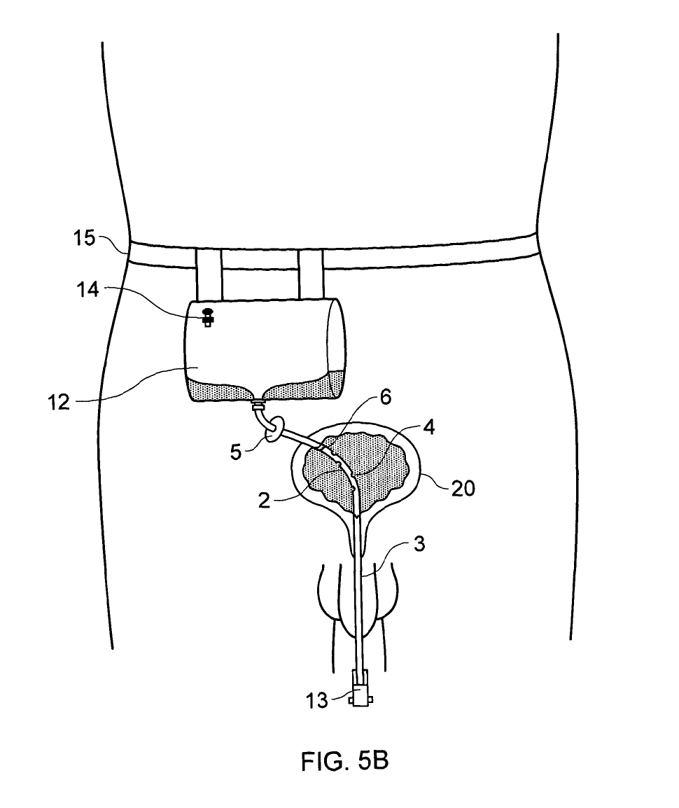

When located within a patient, the abdominal end of the suprapubic section 1 of the catheter first passes through the abdominal stop 5, which grips and retains the catheter in position on the abdominal wall of the user, and then through the abdominal wall along a suprapubic track to join the bladder section 2 which is positioned in the bladder to drain urine. The bladder section leads on to the urethral section 3 which is positioned in the urethra. The location of the catheter within a patient can be seen from FIGS. 5a and 5b.

Referring to FIG. 1 again, the bladder section 2 with the plurality of drainage eyeholes 4 includes a non-return cuff 6 at its upper end which prevents the catheter from extruding retrogradely out of the suprapubic track in the abdominal wall. Small lateral irrigation eyeholes 7 are placed in the catheter tubing of the bladder section positioned within the projecting portion of the cuff to wash out any accumulated debris.

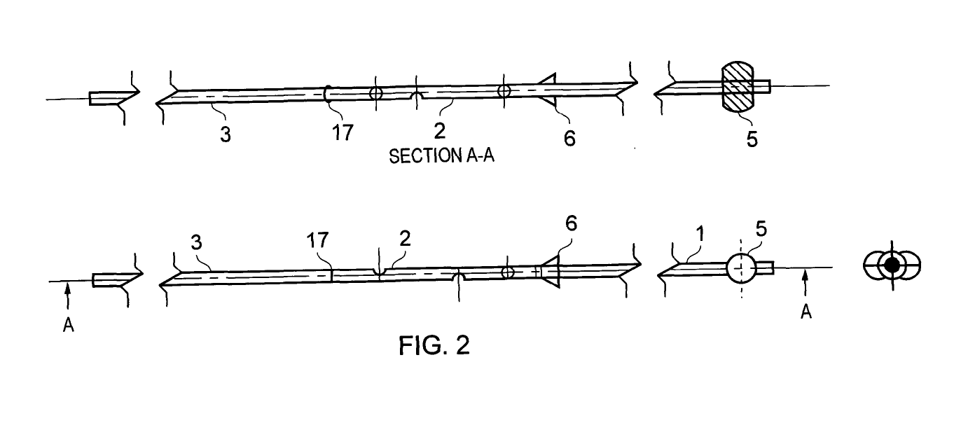

Referring to FIG. 2, the catheter has a non-return cuff 6 of a frusto-conical shape, and an abdominal stop 5. Also shown is a radio-opaque or ultrasound marker 17 at the junction between the bladder section 2 and the urethral section 3.

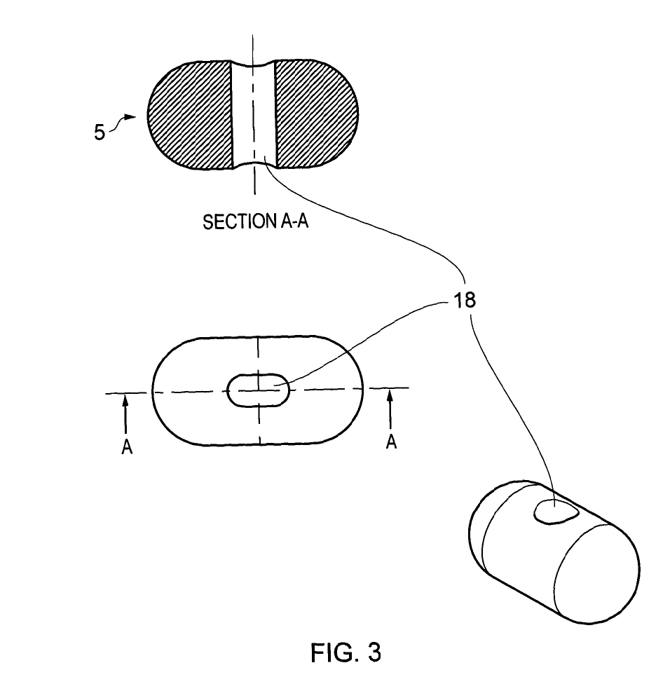

Referring to FIG. 3, there is illustrated one example of an abdominal stop 5. As can be seen, there is an oval channel 18 through the abdominal stop for receiving and gripping the suprapubic section of the catheter.

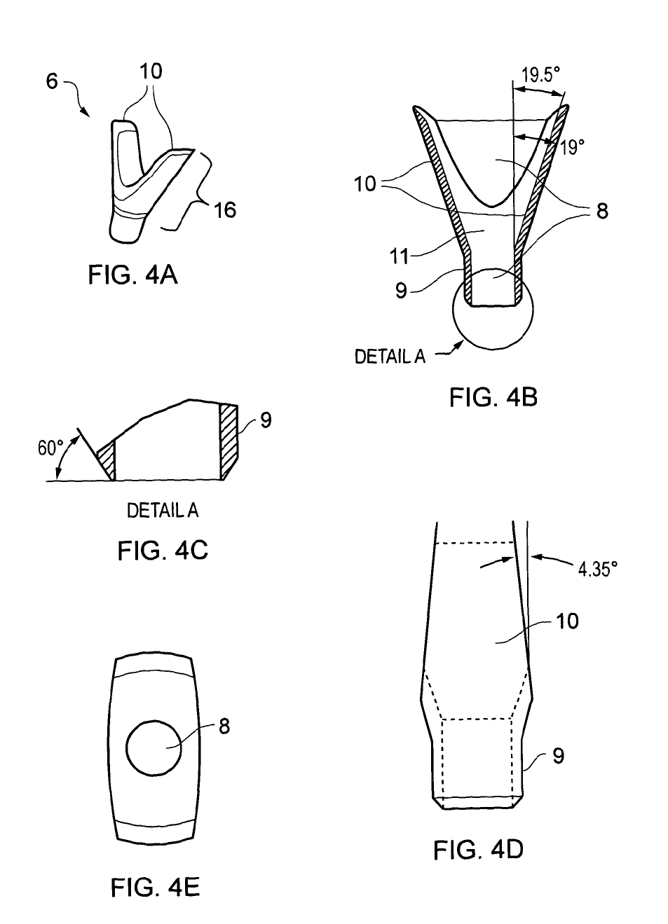

Referring to FIGS. 4a to 4e there is illustrated a non-return cuff 6. FIG. 4a shows a non-return cuff having a projection portion 16 comprising two inclined wings 10. In other words the projection portion is substantially V-shaped. FIG. 4b shows a longitudinal cross-section of the same embodiment of the non-return cuff. As can be seen from FIG. 4b, the cuff has a channel 8 for receiving a catheter. The channel 8 extends throughout the centre of the cuff and between the two inclined wings 10. In other words the two inclined wings 10 are positioned on opposing sides of the channel 8. The inclined wings are joined by a resiliently deformable element 11 which biases the wings apart from each other. As can be seen from FIG. 4b, at the end of the wings, where the distance between the wings is smallest, a sleeve 9 is formed around part of the channel 8. The ends of the sleeve 9, furthest from the wings, taper towards the channel. This is for ease of insertion of the cuff into a bladder along a suprapubic track defined by a catheter held within the channel of the cuff. This tapering can be most clearly seen from FIG. 4c which provides an enlarged view of the area “A” circled in FIG. 4b.

The structural configuration of the cuff is designed to balance the need for flexibility to allow ease of insertion with strength and stiffness to enable its non-return function. In the embodiment shown in FIG. 4d, the wings 10 have a distorted elongated polygonal shape, in which the widest part of the elongated polygon is near the end of the wings being closest to the sleeve, as shown in FIG. 4d. This shape provides the arms with strength to resist deformation away from the channel whilst at the same time minimising any enlargement of the suprapubic track and the aperture into the bladder defined by the catheter on insertion. The wings are also curved, as can be seen from FIG. 4e.

Referring to FIGS. 5a and 5b, there is illustrated a urine collection system.

FIG. 5a shows a catheter having an abdominal stop 5 attached to the suprapubic section 1 and positioned against the abdomen, drainage holes 4 in the bladder section and a non-return cuff 6 attached to the bladder section 2 upstream of the drainage holes 4 (i.e. between the drainage holes and the abdominal stop) and inserted into the bladder 20.

FIGS. 5a and 5b show the upper or suprapubic end of the catheter, beyond the abdominal stop 5, attached to an abdominal urine collection pouch 12 which will hold a volume typically up to 500 ml of urine. There is no valve between the catheter and the pouch. Urine can flow freely from the bladder into the pouch as the bladder fills or if the bladder muscle contracts. When the bladder muscle relaxes, urine can return into the bladder 20. This system allows the bladder 20 to fill normally and if the bladder pressure rises to a threshold pressure, for example as a result of a contraction of the bladder (detrusor) muscle, urine enters the abdominal urine collection pouch and then returns to the bladder once the pressure falls below the threshold, for example as the bladder muscle relaxes. Thus a low pressure urinary reservoir is maintained.

The bladder and any urine collection pouch 12 are emptied by releasing the catheter valve 13 at the lower end of the catheter.

The catheter and the pouch provide a closed urine collection system. A Luer or similar fitting 14 may be included at the top of the pouch (e.g. as shown in FIG. 5b) so that the pouch and catheter can be irrigated if necessary to clear any accumulated debris.

To flush the system, normal saline or other standard bladder washout solutions can be used to irrigate the bladder either through the Luer lock on the abdominal urine collection pouch or through the drainage funnel (not shown) at the upper end of the catheter. By releasing the catheter valve at the lower end of the catheter, the bladder can be flushed on a regular basis, the frequency depending on the amount of debris accumulating in the urine.

As can be seen from the FIG. 5b, the abdominal urine collection pouch 12 is attached to the user's abdomen by a waistband 15.

The manner of introduction of the catheter of the present invention will now be described. A transurethral flexible cystoscopy should initially be performed to exclude the presence of pathology in the urethra or bladder such as stricture, tumour or stones. The urethral end of the ACS catheter is inserted through the suprapubic cystotomy and when this becomes visible in the bladder, the end is held in a pair of endoscopic grabbing forceps. The cystoscope, grabbing forceps and catheter are withdrawn from the urethra.

Gentle traction is applied to the urethral end of the catheter until the non-return cuff on the bladder section of the catheter has been passed through the abdominal wall and entered the bladder. Gentle traction is then applied to the upper or suprapubic end until the non-return cuff engages with the bladder wall thus preventing further withdrawal of the catheter from the bladder. Having reached that point, the abdominal flange is adjusted by sliding it along the catheter until it rests gently against the abdominal wall. In that way the catheter is held in place between the flange on the surface of the body and the non-return cuff within the bladder.

With the catheter in situ, the suprapubic section of the catheter is trimmed and attached to the abdominal urine collection pouch following the instructions provided with the pouch. After trimming the urethral section of the catheter to a suitable length for the patient, a drainage funnel is fitted to the lower end of the catheter and a catheter valve introduced into this.

To change the catheter, one possibility is to detach the abdominal urine collection pouch from the suprapubic section of the catheter above the abdominal flange. The drainage funnel is detached from the urethral end of the catheter. A guidewire is passed through the catheter from the suprapubic end until it emerges from the end of the urethral section. Whilst the guidewire is held securely, the abdominal flange is removed from the suprapubic section by sliding it along to the upper end of the catheter following which the used catheter is removed from the urethral end. The catheter is passed over the guide wire from the suprapubic end until it emerges from the urethra, the guide wire is removed and the same procedure as for the initial catheterisation is followed, securing the non-return cuff in the bladder and adjusting the abdominal flange before attaching the abdominal urine collection pouch at the suprapubic end and drainage funnel at the urethral end. A standard catheter valve is introduced into the drainage funnel.

Alternatively, the abdominal urine collection pouch may be detached from the suprapubic section of the catheter above the abdominal flange. Holding the catheter firmly between the abdominal wall and the abdominal flange, the flange is removed by sliding it off the suprapubic end of the catheter and a small connecting rod is inserted into the lumen at the end of the suprapubic section. The lumen at the urethral end of the new catheter is then inserted over the connecting rod and when firmly attached, gentle traction is applied to the urethral end of the used catheter to pull it together with the new catheter through the abdominal wall, bladder and urethra until it emerges from the lower or urethral end. The used catheter and the connecting rod are removed from the new catheter and the same procedure is followed to introduce the non-return cuff into the bladder, adjust the abdominal flange and attach the abdominal urine collection pouch and drainage funnel into the suprapubic and urethral ends of the new catheter respectively. A standard catheter valve is introduced into the drainage funnel.

The bladder and the urine collection pouch may be emptied by releasing the catheter valve 13 at the lower end of the catheter. To flush the urine collection pouch and bladder, irrigation can be performed through the Luer or similar fitting 14 on the abdominal urine collection pouch using any standard bladder instillation or washout solution and by releasing the catheter valve 13.

The catheter provides a versatile means of draining the bladder. As an alternative to the abdominal urine collection pouch, a drainage funnel can be attached to the upper or suprapubic section of the catheter. With a drainage funnel at both ends, the bladder can be drained or flushed with a standard bladder irrigation fluid from either or both ends or using a catheter valve or a urine collection bag at one end and a spigot at the other. Bladder irrigation using standard bladder washout and other solutions can be performed from either end.

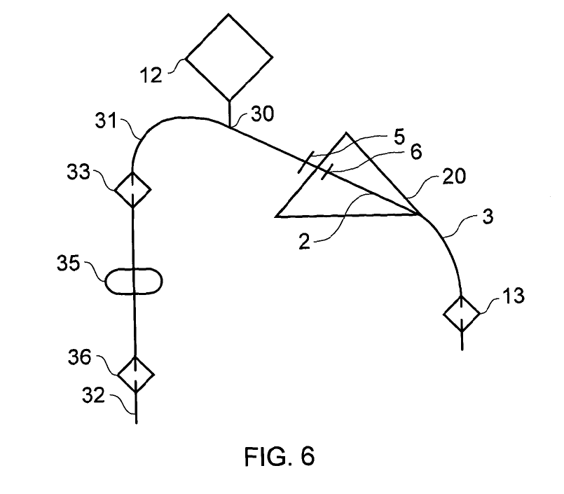

An embodiment of the present invention will now be described with reference to FIG. 6. In the embodiment, the arrangements described above with reference to FIGS. 1 to 5 are modified by the provision of a drainage section of the catheter extending from the suprapubic section to the drainage outlet. FIG. 6 shows the embodiment schematically, but the components corresponding to those described above are indicated by the same reference numerals, and may have similar construction. That construction will not be described in further detail now.

In the embodiment of the invention shown in FIG. 6, a T-junction 30 is provided in the suprapubic section of the catheter, between the abdominal stop 5 and the urine collection pouch 12, with the leg of the “T” leading through the bladder to the catheter valve 13, one arm of the “T” leading to the collection pouch 12 and the other arm of the “T” forming a drainage section 31, leading to a drainage outlet 32. From the T-junction 30, the drainage section 31 has a first valve 33, a urine collection bag 35 and a second valve 36. The drainage section 31 is preferably made of similar materials, etc as the suprapubic, bladder and urethral sections of the catheter, as previously described. It is thus flexible.

The first and second valves 33, 36 are then preferably pinch valves, which act to compress the relevant part of the drainage section 31 to close it. Pinch valves are preferred, because they do not cause contamination of the interior of the drainage section 31, as they act on its exterior. Any suitable pinch valve may be used, but a preferred pinch valve is that disclosed in an article entitled “Design and development of a novel automatic valve system for long-term catheterised urinary incontinence patients” by S. M. Lee et al published in the Proceedings of the Institution of Mechanical Engineers, volume 221, part H: journal of Engineering in Medicine, pages 665 to 676, published 9 May 2007

In that article, the pinch valve was described in which a valve shuttle was moved a by a remote-driven screw towards and away from a stop, with the catheter passing between the shuttle and the stop so that movement of the shuttle towards the stop compressed the catheter, and closed it, and reverse movement of the shuttle opened the catheter to permit urine to be drained. The unit was operated by a micro-controller which permitted time-controlled operation of the motor and shuttle. There was also a manual override facility, operated by a magnetic reed switch. That reed switch could be activated by bringing a magnet close to it, thereby enabling a patient themselves to trigger the control of the valve. Thus, in the arrangement shown in FIG. 6, if such a controller and reed switch is provided for the valves 33, 36, there may be timed control of the valves 33, 36 or the patient may themselves control those valves using e.g. a magnet. Electromagnetic controls for the valves may be an alternative. The article provides further details of the pinch valve, and so those details will not be described in more detail now.

Between the first and second valves 33, 36 is a urine collection bag 35. The purpose of that bag is to permit urine to be collected, and drained at suitable intervals. With the first valve 33 open, and the second valve 36 closed, urine from the bladder 20 and/or the collection pouch 12 may flow to the collection bag 35. At some suitable time e.g. a time convenient for the patient, the second valve 36 may then open to allow urine from the bag 35 to drain via the drainage outlet 32. Thus, the structure can mimic the normal action of the bladder.

The collection bag 35 or pouch 12 may contain a pH sensor. One example of such a sensor is described in WO 2006/000764. That sensor was based on a polymer matrix comprising a chemically bound pH indicator, which indicator only responds to a sustained increase in pH. Again, that pH sensor will not be described in more detail now. It should be noted that other pH sensors may also be used, and the pH sensor may be in the drainage or suprapubic section of the catheter, as well as or as an alternative to being in the collection bag 35 or pouch 12.

The system of the embodiment of FIG. 6 is designed to mimic normal bladder action by providing bladder drainage at regular intervals. Cognitive patients are able to control the drainage valve directly. Alternatively, those with limited manual dexterity could use a remote control mechanism or the drainage intervals could be preset by means of an electronic control system. If the resumption of normal bladder action is achieved, the muscular tone of the bladder and the antibacterial action of the bladder mucosa will be retained.

The controlled drainage may be provided by an electrically operated pinch valve as previously described. The valve can be opened by a patient wearing a magnetic ring on a finger or, in the case of patients unable to operate the valve control, by an electronic timing device which is adjustable over a wide timing range.

A secondary storage reservoir for urine is provided by the pouch 12 for patients suffering from detrusor overactivity. If, as a result of involuntary contraction of the bladder, a pressure surge is generated, the urine is forced into the secondary reservoir. Consequently the risk of kidney reflux and the associated damage is reduced. The maintenance of normal bladder action means that the advantages noted above are retained. If detrusor overactivity is unchecked the drainage of the bladder would occur at intervals too short to allow the bladder to fill.

Involuntary contraction of the bladder would normally cause a pressure surge in the bladder with the consequences outlined above. If, however, the pouch is fitted, the urine will be automatically transferred to the pouch without an excessive rise in bladder pressure. When the bladder subsequently relaxes and the pressure falls, the urine will transfer back into the bladder. It is therefore possible to drain the bladder at the normal intervals and still maintain correct bladder function by emptying completely and slowly filling up again. If, alternatively, the bladder is voided with every surge in bladder pressure, the normal operation of the bladder is lost and, in effect, the bladder is drained continuously.

Detailed investigations have shown the remarkable ability of bacteria to colonise the bladder, with serious consequences, from many different sources. The embodiment of FIG. 6 may be controlled by natural bladder pressures and sealed from the atmosphere as far as possible to reduce bacterial infection to a level that can be restrained by the natural defences of the bladder.

The suprapubic urethral catheter is effectively sealed along the suprapubic and urethral channels and consequently normal bladder pressures are maintained. The pouch 12 and the collection bag 35 are both flexible containers and consequently the pressure within them will not depart from atmospheric pressure in spite of the fact that the volume of urine within them changes. The system is therefore voiding into a region at atmospheric pressure, thereby mimicking normal bladder action.

It is clearly desirable to keep the number of catheter changes to a minimum. The pouch of the proposed catheter system provides a portal for routine maintenance by: (a) bladder irrigation; (b) bladder washout; and (c) bladder instillation.

Bladder irrigation is achieved by administering fluid (usually saline) into the bladder using a 3 way catheter and irrigation set (usually post urological surgery to prevent clot retention).

Bladder washout is achieved by administering a solution (usually saline) manually into the bladder to remove debris using a bladder syringe, alternatively depressing and withdrawing the plunger until the debris is removed.

Bladder instillation: a pre-packed sterile reagent (usually 100 mls) is allowed to drain into the bladder under gravity. The fluid retained in the bladder for a specified period of time (usually 15 minutes) and then allowed to drain out under gravity. Installations are mainly used to dissolve encrustation or reduce infection. Precipitation results from elevation of the pH of the urine by urease-producing bacteria. This has serious consequences not only because of the resulting blockages but also because the bacteria within the struvite stones lead to sustained infection. The catheter system incorporate a pH sensor in the pouch 12, the tubing, or the collection bag 35, e.g. as described above with reference to WO 2006/000764 to give early warning of an elevation in the pH in the urine so that action can be taken before precipitation occurs. The bacteria of Proteus mirabilis convert the urea in the urine into ammonia thereby increasing the pH of the urine. If this process is allowed to continue unchecked, precipitation and the formation of encrusting biofilms will take place. It is essential for the welfare of the patient that action is taken before precipitation occurs.

The new catheter has a multiplicity of drainage holes 4, an arrangement which offers two advantages:

(i) an increase in the effective drainage area into the catheter with a resulting reduction in the pressure difference across the drainage holes. As a result the risk of damage to the bladder mucosa and the formation of pseudo polyps is reduced.
(ii) At the end of the bladder drainage process the sudden cessation of flow results in a rapid loss of kinetic energy by the urine in the drainage tube. As a result the transitory suction can create negative pressures of between 150 and 350 cm of water. This often causes pain for the patient and damage to the bladder mucosa. The provision of a multiplicity of drainage holes should ensure that, during the final stages of drainage, the flow velocity is reduced with a corresponding reduction in the kinetic energy of the arrested fluid.

It has been shown that bacteria, which are microorganisms can invade the catheter system through extremely narrow gaps, for example, the adjacent cones of a cone tap. In an effort to counter the invasion, pinch valves are used whenever possible.

In the arrangement shown in FIG. 5b the urine collection pouch 12 is immediately accessible, but this has the problem that if there was an impact on the urine collection pouch 12 when the urine collection pouch 12 was filled, e.g. because the patient fell forward, the resulting pressure wave in the urine collection pouch 12 could lead to reflux of urine into the kidneys or damage to the bladder. Therefore, it is desirable that the urine collection pouch 12 is protected by a cover. A cover should protect the urine collection pouch 12 from compression, this must also be of a suitable shape and easy to remove.

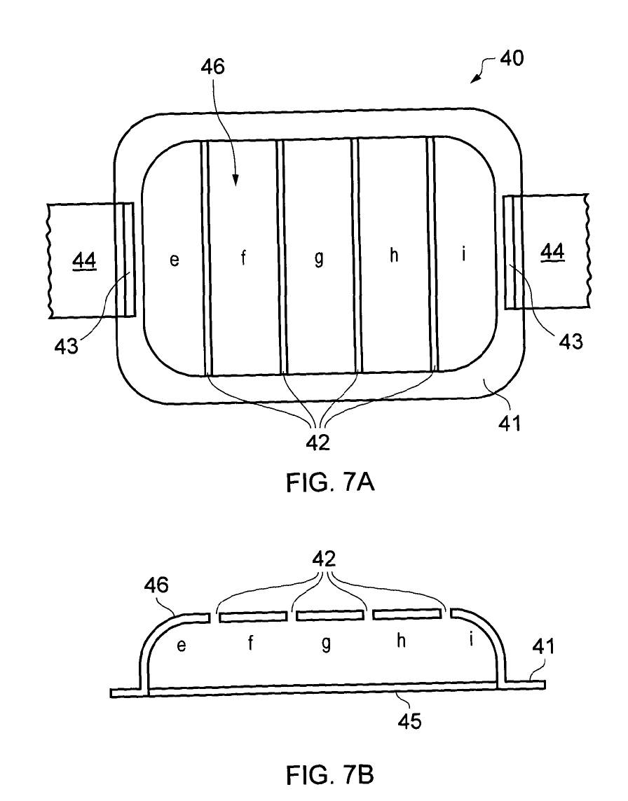

FIGS. 7a and 7b show a protective cover 40 which may be used to protect the urine collection pouch 12, when that pouch 12 is used in the present invention. As shown in FIGS. 7a and 7b, the cover 40 comprises a flange 41 surrounding an opening 45. The opening 45 is then covered by a domed wall 46 so that the abdominal collection pouch 12 fits in the space defined within the wall 46, through the opening 45. The wall 46 has openings 42 therein to allow the wall 40; and hence the cover 40 to be deformed, by deformation of the flange 41, to allow it to follow the contours of the body. Thus, there are sections e, f, g, h and i in the wall 46 which form arches over the pouch and are retained in place due to their attachment to the flange 41.

A waist band 44 may be provided to enable the patient to fit the cover 40 in place, with the ends of the waist band 44 passing through slots 43 in the flange 41. The waist band 44 may itself be connected to, or part of, the waist band 15 used to support the urine collection pouch 12.

In the arrangements described above, the catheter is held in place by the abdominal stop 5 and the non-return cuff 6 which non-return cuff 6 has two inclined wings 10. However, other arrangements for holding the catheter in the bladder may be used within the present invention.

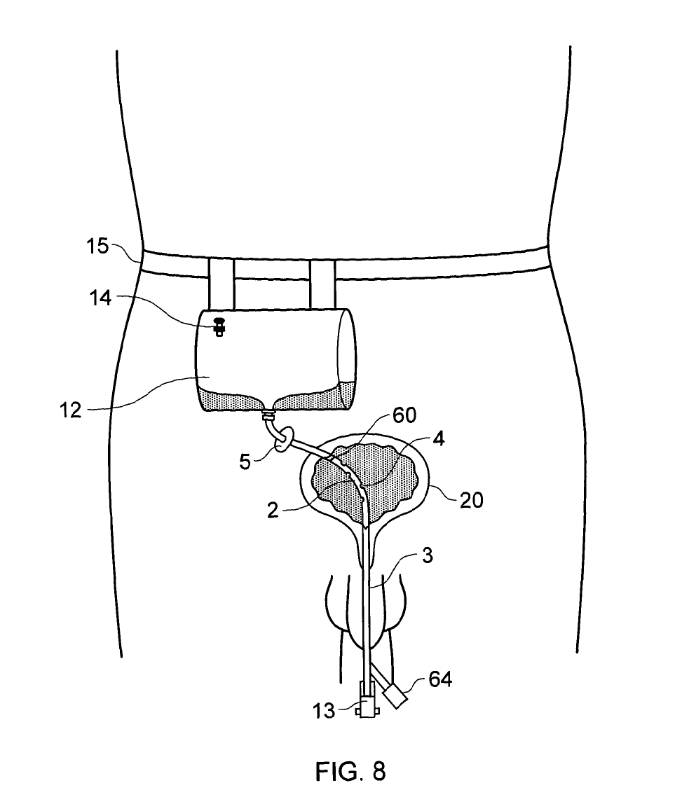

In particular, FIG. 8 shows an arrangement in which a Foley catheter is used. The arrangement is similar to that shown in FIG. 5b, except that the winged non-return cuff 6 is replaced with a non-return cuff in the form of an inflatable spherical balloon 60. That balloon 60 is connected via a channel (not shown) in the catheter to a non-return valve 64. By injecting a fluid, such as water, through the non return valve 64, the balloon 60 can be inflated as the fluid passes up the channel to the balloon 60 from the non-return valve 64. The channel may be moulded into the wall of the urethral and bladder sections 3, 2 of the catheter.

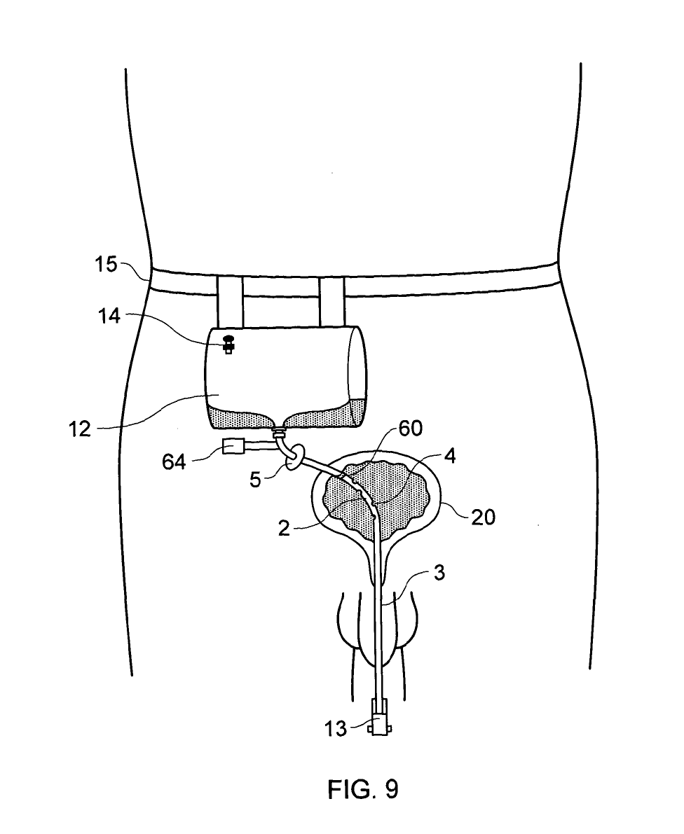

The arrangement shown in FIG. 8 uses urethral filling of the balloon 60. However, it is also possible to have an alternative arrangement, shown in FIG. 9, in which the non return valve 64 is above the abdominal stop 5. Again, that non return valve 64 is connected to the balloon 60 via a channel in the abdominal part of the catheter. In such arrangement, it may be necessary to modify the abdominal stop 5 to enable it to clip over the catheter.

Note that, in the arrangement shown in FIGS. 8 and 9, other features which are the same as those in FIG. 5b are indicated by the same reference numerals, and will not be described in more detail now. Furthermore, the catheter arrangement shown in FIGS. 8 and 9 may be modified by the provision of a drainage section, as in FIG. 6.

Note that, in the arrangements described in FIGS. 8 and 9, it is not necessarily that the balloon 60 is spherical, provided it expands sufficiently to hold the catheter in place within the bladder. The procedure for introduction of the catheter is unchanged from that described earlier. To remove the catheter, it is necessary first to deflate the balloon 60 e.g. by cutting the connection to the non-return valve 64. Use of such a balloon 60 has a further advantage that the pressure it exerts on the bladder wall may be adjustable by introduction of e.g. an air bubble into the liquid in the balloon. It is also desirable that the section of the catheter between the bladder and the urine collection pouch 12 or drainage section 31 is made of non-kink tubing, preferably with a helical wall structure.

### REFERENCES

1. Saint S, Elmore J G, Sullivan S D, Emerson S S, Koepsell T D. The efficacy of silver alloy-coated urinary catheters in preventing urinary tract infection: a meta-analysis. Am J Med 1998: 105:236-241).
2. Maki D G, Tambyah P A. Engineering Out the Risk for infection with Urinary Catheters. Emerging Infectious Diseases 2001: 7(2):342-347.
3. Saint S. Clinical and economic consequences of nosocomial catheter-related bacteriuria. Am J Infect Control 2000: 28(1):68-73.
4. Rosenthal V D. Device-associated nosocomial infections in limited-resources countries: findings of the International Nosocomial Infection Control Consortium (INICC). Am J Infect Control 2008: 36(10):S171-12.
5. Foley FEB. A Self-Retaining Bag Catheter. J Urol 1937: 38:140-143.
6. Warren, J W Catheter-associated urinary tract infections Infect Dis North Am 11 (3): 609-622 1997.
7. Kohler-Ockmore J. and Feneley R C L. Long-term catheterisation of the bladder: prevalence and morbidity. British Journal of Urology. 77: 347-351 1996
8. Hellstrom L, Ekeland P, Milsom I, and Melistrom D. Prevalence of incontinence: use of aids. Age and Ageing. 19: 383-389 1990.
9. Kunin, C M. Can we build a better urinary catheter? N Eng J Med 319 (6):365-366, 1988.

1. A urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, the connection forming a closed valve-free fluid flowpath from the bladder section to the urine collection container and from the urine collection container to the bladder section, whereby the urine collection container provides a low pressure reservoir, and wherein the catheter further comprises a drainage section extending from the suprapubic section to a drainage outlet, the drainage section having at least one valve therein.

2. A catheter according to claim 1, wherein said at least one valve is a pinch valve.

3. A catheter according to claim 1 wherein said at least one valve has an moveable actuator for engaging said drainage section, and a timer for controlling timed actuation of said moveable actuator.

4. A catheter according to claim 1 wherein said at least one valve has a moveable actuator for engaging said drainage section and there is a user-activatable control for controlling actuation of said moveable actuator.

5. A catheter according to claim 4, wherein said user-activatable control is remote from said pinch valve and magnetically or electromagnetically connected thereto, said user-activatable control being arranged to generate magnetic or electromagnetic signals for activating said moveable actuator.

6. A catheter according to claim 1, wherein the drainage section further includes a urine collection bag.

7. A catheter according to claim 6 wherein said at least one valve comprises a first valve between said suprapubic section and said urine collection bag, and a second valve between said urine collection bag and said drainage outlet.

8. A urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, the connection providing a flowpath from the bladder section to the container, and thus providing a low pressure reservoir and wherein the catheter comprises a drainage section extending from the suprapubic section to a drainage outlet, the drainage section including a urine collection bag.

9. A catheter according to claim 7, wherein said urine collection bag and/or said urine collection container contains a pH sensor.

10. A urine collection system comprising:
a suprapubic urethral catheter comprising a hollow tube having sequentially, a suprapubic section, a bladder section and a urethral section, wherein, the bladder section comprises one or more drainage holes in the wall of the tube and there is an abdominal stop attached to the suprapubic section; and
a urine collection container connected to the suprapubic section, the connection forming a closed valve-free fluid flowpath from the bladder section to the urine collection container and from the urine collection container to the bladder section, whereby the urine collection container provides a low pressure reservoir;
wherein the catheter comprises a drainage section extending from the suprapubic section to a drainage outlet, and said drainage section and/or the suprapubic section has a pH sensor therein.

11-44. (canceled)
